(19) United States
(12) Patent Application Publication
(10) Pub. No.: US 2017/0071902 A1
(43) Pub. Date: Mar. 16, 2017

(54) NOX INHIBITOR AND NFkB INHIBITOR CONTAINING METHOXYFLAVONE

(71) Applicant: SUNTORY HOLDINGS LIMITED, Osaka (JP)

(72) Inventors: Daisuke TAKEMOTO, Osaka (JP); Yoshiko ONO, Osaka (JP); Sumio ASAMI, Tokyo (JP); Satomi SHIMOYOSHI, Osaka (JP)

(21) Appl. No.: 15/308,868

(22) PCT Filed: May 1, 2015
(86) PCT No.: PCT/JP2015/063100

§ 371 Date: Nov. 4, 2016

(30) Foreign Application Priority Data
May 9, 2014 (JP) 2014-097875

(51) Int. Cl. A61K 31/353

(52) U.S. Cl. CPC A61K 31/353

(57) ABSTRACT

The present invention aims at providing NOX inhibitors and NFκB inhibitors having superior actions, as well as agents for preventing or treating NOX- or NFκB-associated diseases that utilize such inhibitors. To this end, specified methoxyflavones are employed.

### TECHNICAL FIELD

The present invention relates to NOX inhibitors and NFκB inhibitors containing specified methoxyflavones, as well as uses thereof.

### BACKGROUND ART

NADPH oxidase (NOX), typically occurring in basophils, is an enzyme known to generate O²⁻. Hence, inhibiting NOX is believed to be useful in preventing or treating a variety of diseases associated with oxidation in vivo.

NFκB is a transcription factor that regulates the transcription of genes encoding various molecules such as proinflammatory cytokines (e.g. TNFα, IL-1β, IL-6), chemokines (e.g. IL-8, MIP1α), and inducible effector enzymes (e.g. iNOS and COX-2) and it plays an important role in inflammatory reactions. It has been shown that activation of the NFκB pathway can lead to a variety of inflammatory diseases.

Some of the methoxyflavones contained in plant extracts have been reported to possess an antioxidizing action. Many of those methoxyflavones have hydroxyl groups (Non-Patent Documents 1-3).

Black ginger (Kaempferia parviflora) is a plant belonging to the Zingiberaceae family and is also known as black turmeric in Japan. Black ginger is a traditional herbaceous plant in Thailand where it is also known as Kra chai dahm. Black ginger is known to contain not only methoxyflavones that have hydroxyl groups but also those which have no hydroxyl group (Non-Patent Document 4).

### CITATION LIST

### Non-Patent Literature

Non-Patent Document 1: Biochem Pharmacol., 2012, 84(2), 182-191
Non-Patent Document 2: Free Radic Biol Med., 1999, 27(1-2), 95-99
Non-Patent Document 3: Biochem Pharmacol., 1987, 36(5), 717-720
Non-Patent Document 4: “The Structures of Components in A Plant of the Zingiberaceae Family, Kaempferia parviflora, as well as Their α-Glucosidase Inhibitory Activities and Antimutagenicities”, a doctor's thesis of Mr. Toshiaki Azuma, Graduate School of Human Life Science, Osaka City University

### SUMMARY OF INVENTION

### Technical Problem

An object of the present invention is to provide NOX inhibitors and NFκB inhibitors having superior actions. Another object is to provide agents for preventing or treating diseases associated with NOX or NFκB.

### Solution to Problem

The present inventor conducted intensive studies to attain these objects and found as a result that specified methoxyflavones obtained from black ginger had superior NOX inhibiting action and/or NFκB inhibiting action.

Briefly, the present invention relates to, but is not limited to, the following.

[1] NOX inhibitor containing at least one methoxyflavone having a structure represented by the following formula (I):

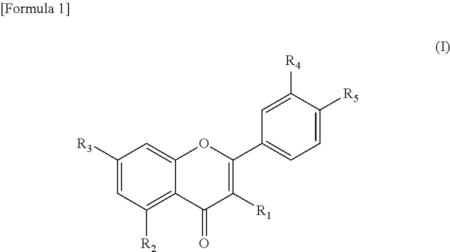

(where R₁, R₄ and R₅ are each independently hydrogen or a methoxy group, and R₂ and R₃ are each a methoxy group).
[2] The NOX inhibitor according to [1], wherein the at least one methoxyflavone is selected from group A consisting of 5,7,3′,4′-tetramethoxyflavone, 3,5,7,3′,4′-pentamethoxyflavone, 5,7-dimethoxyflavone, and 5,7,4′-trimethoxyflavone.
[3] The NOX inhibitor according to [2], wherein a ratio of the total content of said methoxyflavones of group A to the total contents of said methoxyflavones of group A and methoxyflavones of group B consisting of 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which is expressed as A/(A+B), exceeds 0.65 on a molar basis.
[4] An agent for preventing or treating an NOX-associated disease that contains said at least one methoxyflavone as defined in [1] or [2].
[5] The agent according to [4], wherein the NOX-associated disease is selected from the group consisting of allergic diseases, Parkinson's disease, cerebral infarction, cataract, epilepsy, spinal cord injury, arteriosclerosis, retinopathy of prematurity, renal disorder, peptic ulcer, pancreatitis, ulcerative colitis, myocardial infarction, adult respiratory distress syndrome, pulmonary emphysema, collagen diseases such as chronic rheumatoid arthritis, angiitis, edema, complications of diabetes, ultraviolet disorders, altitude sickness, porphyria, burns, frostbite, contact dermatitis, shock, failure of multiple organs, DIC, cancer, aging, fatigue, sarcopenia (progressive decline in skeletal muscle mass), mitochondrial dysfunction, dementia, and Alzheimer's disease.
[6] The agent according to [4] or [5], wherein a ratio of the total content of said methoxyflavones of group A as defined in [2] to the total contents of said methoxyflavones of group A and said methoxyflavones of group B as defined in [3], which is expressed as A/(A+B), exceeds 0.65 on a molar basis.
[7] NFκB inhibitor containing at least one methoxyflavone having a structure represented by the following formula (I):

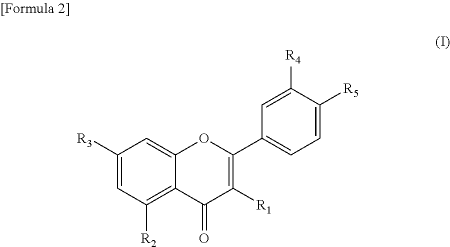

(where R₁, R₄ and R₅ are each independently hydrogen or a methoxy group, and R₂ and R₃ are each a methoxy group).
[8] The NFκB inhibitor according to [7], wherein the at least one methoxyflavone is selected from group A′ consisting of 5,7,3′,4′-tetramethoxyflavone, 5,7-dimethoxyflavone, and 5,7,4′-trimethoxyflavone.
[9] The NFκB inhibitor according to [8], wherein a ratio of the total content of said methoxyflavones of group A′ to the total contents of said methoxyflavones of group A′ and methoxyflavones of group B′ consisting of 3,5,7,3′,4′-pentamethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which is expressed as A′/(A′+B′), exceeds 0.48 on a molar basis.
[10] An agent for preventing or treating an NFκB-associated disease that contains said at least one methoxyflavone as defined in [7] or [8].
[11] The agent according to [10], wherein the NFκB-associated disease is selected from the group consisting of rheumatoid arthritis, inflammatory colitis, osteoarthritis, osteolysis, tendinitis, sciatica, herniated disc, stenosis, myelosis, low back pain, zygapophyseal joint pain, carpal tunnel syndrome, tarsal tunnel syndrome, failed back surgery syndrome, AIDS, arteriosclerosis, asthma, arthritis, diabetes, inflammatory colitis, hepatitis, stroke, dementia, muscle wasting, viral infection, skin aging including photoaging, cancer, and aging.
[12] The agent according to [10] or [11], wherein a ratio of the total content of said methoxyflavones of group A′ as defined in [8] to the total contents of said methoxyflavones of group A′ and said methoxyflavones of group B′ as defined in [9], which is expressed as A′/(A′+B′), exceeds 0.48 on a molar basis.
[13] NOX inhibitor containing 3′,4′-dimethoxyflavone.
[14] An agent for preventing or treating an NOX-associated disease that contains 3′,4′-dimethoxyflavone.
[15] The agent according to [14], wherein the NOX-associated disease is selected from the group consisting of allergic diseases, Parkinson's disease, cerebral infarction, cataract, epilepsy, spinal cord injury, arteriosclerosis, retinopathy of prematurity, renal disorder, peptic ulcer, pancreatitis, ulcerative colitis, myocardial infarction, adult respiratory distress syndrome, pulmonary emphysema, collagen diseases such as chronic rheumatoid arthritis, angiitis, edema, complications of diabetes, ultraviolet disorders, altitude sickness, porphyria, burns, frostbite, contact dermatitis, shock, failure of multiple organs, DIC, cancer, aging, fatigue, sarcopenia (progressive decline in skeletal muscle mass), mitochondrial dysfunction, dementia, and Alzheimer's disease.

### Advantageous Effects of Invention

The present invention is capable of providing NOX inhibitors and NFκB inhibitors having superior actions. Utilizing such inhibitors, the present invention is also capable of providing agents for preventing or treating NOX- or NFκB-associated diseases.

Unlike those which have heretofore been found to benefit from antioxidizing and other actions, the methoxyflavones that are selectively used in the present invention have no hydroxyl group and yet exhibit superior NOX inhibiting and other actions.

### DESCRIPTION OF EMBODIMENTS

### Methoxyflavones

In the present invention, at least one, preferably at least two, more preferably at least three, more preferably at least four, more preferably at least five, more preferably at least six, more preferably at least seven, and even more preferably at least eight of methoxyflavones are used that have a structure represented by the following formula (I):

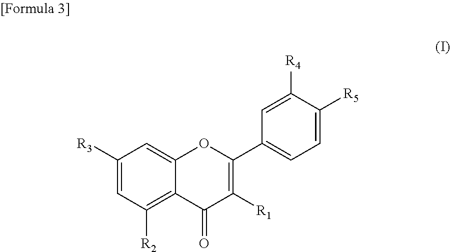

(where R₁, R₄ and R₅ are each independently hydrogen or a methoxy group, and R₂ and R₃ are each a methoxy group).

In inhibiting NOX or in treating or preventing NOX-associated diseases according to the present invention, said at least one methoxyflavone is preferably selected from group A consisting of 5,7,3′,4′-tetramethoxyflavone, 3,5,7,3′,4′-pentamethoxyflavone, 5,7-dimethoxyflavone, and 5,7,4′-trimethoxyflavone. The NOX inhibitor or the agent for treating or preventing NOX-associated diseases may contain not only the methoxyflavones of group A but also other compounds, say, at least one methoxyflavone derived from black ginger as selected from group B consisting of 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone. It should, however, be noted that methoxyflavones of group A exhibit higher NOX inhibiting action than methoxyflavones of group B. Hence, methoxyflavones of group A are preferably contained in higher proportions. For example, a ratio of the total content of the methoxyflavones of group A to the total contents of the methoxyflavones of groups A and B, which is expressed as A/(A+B), is preferably greater than 0.65, more preferably 0.66 and above, more preferably 0.67 and above, more preferably 0.68 and above, more preferably 0.69 and above, more preferably 0.70 and above, and even more preferably 0.71 and above, on a molar basis (or weight basis). The above-defined ratio has no upper limit and may be 1.00 and below.

In inhibiting NFκB or in treating or preventing NFκB-associated diseases according to the present invention, said at least one methoxyflavone is preferably selected from group A′ consisting of 5,7,3′,4′-tetramethoxyflavone, 5,7-dimethoxyflavone, and 5,7,4′-trimethoxyflavone. The NFκB inhibitor or the agent for treating or preventing NFκB-associated diseases may contain not only methoxyflavones of group A′ but also other compounds, say, at least one methoxyflavone derived from black ginger as selected from group B′ consisting of 3,5,7,3′,4′-pentamethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone. It should, however, be noted that the methoxyflavones of group A′ exhibit higher NFκB inhibiting action than the methoxyflavones of group B′. Hence, the methoxyflavones of group A′ are preferably contained in higher proportions. For example, a ratio of the total content of the methoxyflavones of group A′ to the total contents of the methoxyflavones of groups A′ and B′, which is expressed as A′/(A′+B′), is preferably greater than 0.48, more preferably 0.49 and above, more preferably 0.50 and above, more preferably 0.51 and above, more preferably 0.52 and above, more preferably 0.53 and above, more preferably 0.54 and above, more preferably 0.55 and above, and even more preferably 0.60 and above, on a molar basis (or weight basis). The above-defined proportion has no upper limit and may be 1.00 and below.

Alternatively, the methoxyflavone is 3′,4′-dimethoxyflavone.

Most of the methoxyflavones mentioned above can, for example, be obtained from black ginger (Kaempferia parviflora) in accordance with the method described in Non-Patent Document 4. Alternatively, they can also be obtained by, for example, the method detailed in Example 1 of the subject specification. Black ginger is a plant belonging to the Zingiberaceae family and easily available since it grows naturally in Southeast Asia and other regions. It is known that 3′,4′-dimethoxyflavone can be extracted, for example, from Lawsonia alba (hena) using a solvent. See, for example, Phytochemistry Letters, 2011, 4, 454-458.

The types and quantities of the methoxyflavones to be contained in the aforementioned inhibitors and agents can be adjusted as required on the basis of known methods. For example, particular methoxyflavones may be removed using known purification methods or, alternatively, purified forms of particular methoxyflavones may be added to the aforementioned inhibitors or the agents.

The present invention also permits utilization of oil or fat extracts that are obtained from black ginger and which contain the methoxyflavones of formula (I). Such oil or fat extracts are extracts as obtained from black ginger through extraction with oil or fat. The extracts contain methoxyflavones and are reduced in the intensity of a black purple color which is characteristic of black ginger extracts. The extracts may further contain oils or fats, especially those used in the extraction step.

Said oil or fat extracts are considered to differ in terms of the kinds of components contained, their proportions, etc. from extracts that are not derived from black ginger or from extracts that start from black ginger but which have not passed through extraction with oil or fat. For instance, extracts obtained from plants other than black ginger may contain methoxyflavones but their kinds and proportions would differ from those in the oil or fat extracts. Even if black ginger is used as the starting material, the types and proportions of methoxyflavones in extracts that are obtained from it by methods other than extraction with oil or fat, say, extraction with a hydrous alcohol are different from those in the oil or fat extracts. As for the extraction to be performed with oil or fat, it may be applied directly to black ginger or indirectly, for example, to a liquid extract as obtained from black ginger using a solvent other than fat or oil, say, water, a hydrophilic solvent or a mixture thereof.

Said oil or fat extracts contain at least one methoxyflavone of formula (I). The methoxyflavone is preferably selected from group A. The extracts may further contain methoxyflavones selected from group B. The oil or fat extracts preferably contain at least one methoxyflavone selected from among the eleven members of groups A and B, more preferably at least two, more preferably at least three, more preferably at least four, more preferably at least five, more preferably at least six, more preferably at least seven, more preferably at least eight, more preferably at least nine, more preferably at least ten, and even more preferably eleven such methoxyflavones.

The oils or fats that can be employed to produce the oil or fat extracts and which may be contained in such extracts are not particularly limited as long as they are capable of dissolving methoxyflavones. Typically, such oils or fats are at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perilla frustescens var. frustescens oil. The term “middle-chain fatty acid” as used in connection with the middle-chain fatty acid triglycerides means fatty acids having 8 to 12 carbon atoms. At least one, preferably at least two, more preferably three of the fatty acid moieties that constitute said triglycerides are middle-chain fatty acids.

The oil or fat extracts are reduced in the intensity of a black purple color. This can be effectively confirmed by measuring the light absorbance of the extracts.

Specifically, a solution of the extract is prepared in which the total content of the eleven methoxyflavones of groups A and B is 5.0 mg/ml and the solution is measured for its absorbance at a wavelength of 660 nm. The thus measured absorbance is 0.10 or below in the oil or fat extracts. The absorbance is preferably 0.07 or below, more preferably 0.05 or below. Unless otherwise specified, the term “absorbance” as used herein means a value for the case where the cell length (optical path length) is 10 mm. If the cell length of the device used for measurement is not 10 mm, the measured value of absorbance is converted to the value for the case of cell length=10 mm. It is also necessary to use a suitable blank for absorbance measurement.

Hereinafter, processes for producing the oil or fat extracts are described.

Such processes, for example, comprise contacting a plant body of black ginger with an oil or fat and extracting one or more methoxyflavones. A typical example of this method is described below.

First, a plant body of black ginger is provided. This plant body or part of it is dried and ground depending on the need. Subsequently, the plant body or its part is brought into contact with an oil or fat and subjected to extraction. The conditions for extraction are not particularly limited as long as they are capable of extracting methoxyflavones. Typical extraction temperatures are 50 to 180° C., 70 to 170° C., 70 to 150° C., 100 to 150° C., or 120 to 150° C. The extraction time is typically one minute to a day, 10 minutes to 10 hours, or 15 minutes to 5 hours. And the volume of the oil or fat to be used is typically 0.1 to 30 times or 0.5 to 15 times the weight of black ginger. Examples of the oil or fat to be used are as already described above.

Although not wishing to be bound by any theory, the present inventor assumes that in the process of this extraction, methoxyflavones transfer into the oil or fat whereas the components that are responsible for the black purple color of black ginger will remain in the plant body of black ginger. It is also assumed that the components responsible for the distinctive flavor of black ginger will remain in its plant body without transferring into the oil or fat.

Subsequently, after extraction has been performed, the oil or fat extract as obtained by the extraction is optionally freed of insoluble solids by filtration or centrifugation.

Alternatively, the production of an oil or fat extract comprises contacting a plant body of black ginger with water, a hydrophilic solvent or a mixture thereof and extracting at least one or more methoxyflavones, followed by bringing an intermediate extract as obtained by the extraction into contact with an oil or fat and extracting said one or more methoxyflavones. A typical example of this method is described below.

First, a plant body of black ginger is provided as described above. Subsequently, the plant body or part of it is brought into contact with water, a hydrophilic solvent or a mixture thereof and subjected to extraction. The conditions for extraction are not particularly limited as long as they are capable of extracting methoxyflavones. Typical extraction temperatures are room temperature to reflux temperature, 40° C. to reflux temperature, 50° C. to reflux temperature, or at reflux temperature, preferably 50° C. to reflux temperature or at reflux temperature. The extraction time is typically one minute to a day, 10 minutes to 10 hours, or 15 minutes to 5 hours. And the volume of the water, hydrophilic solvent or mixture thereof to be used is typically 0.1 to 30 times or 0.5 to 15 times the weight of black ginger. The hydrophilic solvent to be used is preferably a C₁₋₃ alcohol and/or acetone, more preferably ethanol. For example, 50-100 v/v % ethanol may be used as an extraction solvent. An intermediate extract as obtained by this extraction step is subjected to the next step of extraction with an oil or fat.

In the oil or fat extraction step, the intermediate extract is contacted with an oil or fat to perform extraction. The conditions for extraction are not particularly limited as long as they are capable of extracting methoxyflavones. The extraction temperature is not particularly limited and the process may be performed at, for example, 5° C. and above, or 10° C. and above, or 20° C. and above, or 30° C. and above, or 40° C. and above, or 50° C. and above. The extraction temperature has no particular upper limit and may be of any that does not exceed the reflux temperature of the water, hydrophilic solvent or mixture thereof. The extraction time is typically one minute to a day, 10 minutes to 10 hours, or 15 minutes to 5 hours. And the volume of the oil or fat to be used is typically 0.01 to 30 times or 0.5 to 15 times the weight of black ginger. Examples of the oil or fat to be used are as already described above.

Further, depending on the case, the water, hydrophilic solvent or mixture thereof is evaporated from the intermediate extract before it is contacted with the oil or fat and/or while they are kept in contact with each other. The evaporation may be performed at ordinary or reduced pressure. In the case of performing such positive evaporation, the extraction time is not very important. Presumably, as evaporation proceeds and the amount of the water, hydrophilic solvent or mixture thereof decreases, methoxyflavones will transfer into the oil or fat, occasionally together with the hydrophilic solvent and the like.

Although not wishing to be bound by any theory, the present inventor assumes that in the process of oil or fat extraction, methoxyflavones transfer into the oil or fat whereas the components that are responsible for the black purple color of black ginger will not.

Subsequently, after extraction has been performed, the oil or fat containing extract as obtained by the extraction is optionally freed of insoluble solids by filtration or centrifugation. This also applies to the intermediate extract.

According to the two methods described above, an oil or fat containing extract can be obtained. This extract may be used without further purification but, if necessary, it may be purified. For example, the oil or fat containing extract may be subjected to a step of further extraction to remove the oil or fat. Specifically, the oil or fat containing extract is contacted with water, a hydrophilic solvent or a mixture thereof to extract one or more methoxyflavones. In the process, if there is a need, a solvent of low polarity such as a C₁₋₈ hydrocarbon like n-hexane may be added to the oil or fat containing extract.

Examples of the hydrophilic solvent or its mixture with water to be used are as described above. The extraction temperature is not particularly limited and the process may be performed at, for example, 5° C. and above, or 10° C. and above, or 20° C. and above, or 30° C. and above, or 40° C. and above, or 50° C. and above. The extraction temperature has no particular upper limit and may be of any that does not exceed the reflux temperature of the water, hydrophilic solvent or mixture thereof. The extraction time is typically one minute to a day, 10 minutes to 10 hours, or 15 minutes to 5 hours. And the volume of the water, hydrophilic solvent or mixture thereof to be used is typically 0.01 to 30 times or 0.5 to 15 times the weight of the oil or fat extract.

Further in addition, during the process of extracting methoxyflavones, there is obtained a two-phase mixture of an oil or fat phase derived from the oil or fat containing extract and a phase derived from the water, hydrophilic solvent or mixture thereof, and this mixture is subjected to liquid-liquid separation. As a result, the oil or fat phase can be separated from the phase of the water, hydrophilic solvent or mixture thereof (which is an extract containing methoxyflavones and the solvent). For liquid-liquid separation, the two-phase mixture may, for example, be simply left to stand or it may be subjected to centrifugation. Subsequently, the separated extract is recovered.

The separated extract is in the form of a liquid containing methoxyflavones and the solvent. This liquid may be directly put to use or, alternatively, the solvent (water, hydrophilic solvent or mixture thereof) may be removed to yield an extract in powder form that contains methoxyflavones. The method for removing the solvent is not particularly limited and examples include distillation under ordinary or reduced pressure, freeze-drying, and so on.

In the extract thus freed of oil or fat, methoxyflavones which are characteristic of black ginger are contained at comparatively high concentrations. If necessary, this extract may also be subjected to further purification.

The thus obtained oil or fat extracts have comparatively higher values of the above-defined ratios, A/(A+B) and A′/(A′+B′), than the extracts obtained using a hydrophilic solvent such as ethanol. The preferred ranges for said proportions in the oil or fat extracts are as set forth above.

(NOX Inhibitors)

The present inventor has found that methoxyflavones of formula (I) are effective as NADPH oxidase (NOX) inhibitors. Therefore, one aspect of the present invention relates to a NOX inhibitor or a composition for inhibiting NOX that contains at least one methoxyflavone of formula (I) (in the subject specification, the terms “NOX inhibitor” and “composition for inhibiting NOX” are used interchangeably and unless otherwise specified, when one of these terms is used, the other term shall also be intended.) In another mode, the present invention also relates to a method for inhibiting NOX that comprises administering at least one methoxyflavone of formula (I) to a subject. Alternatively, in place of or in addition to methoxyflavones of formula (I), 3′,4′-dimethoxyflavone may be employed.

Inhibiting NOX leads to preventing or treating NOX-associated diseases. Therefore, another aspect of the present invention relates to an agent for preventing or treating NOX-associated diseases or a composition for preventing or treating NOX-associated diseases that contains at least one methoxyflavone of formula (I) (in the subject specification, the terms “agent for preventing or treating NOX-associated diseases” and “composition for preventing or treating NOX-associated diseases” are used interchangeably and unless otherwise specified, when one of these terms is used, the other term shall also be intended.) In another mode, the present invention relates to a method for preventing or treating NOX-associated diseases that comprises administering at least one methoxyflavone of formula (I) to a subject. Such diseases include: allergic diseases such as atopic dermatitis, allergic rhinitis (pollinosis), allergic conjunctivitis, allergic gastroenteritis, bronchial asthma, infantile asthma, food allergy, drug allergy and hives; Parkinson's disease; cerebral infarction; cataract; epilepsy; spinal cord injury; arteriosclerosis; retinopathy of prematurity; renal disorder; peptic ulcer; pancreatitis; ulcerative colitis; myocardial infarction; adult respiratory distress syndrome; pulmonary emphysema; collagen diseases such as chronic rheumatoid arthritis; angiitis; edema; complications of diabetes; ultraviolet disorders; altitude sickness; porphyria; burns; frostbite; contact dermatitis; shock; failure of multiple organs; DIC; cancer; aging; fatigue; sarcopenia (progressive decline in skeletal muscle mass); mitochondrial dysfunction; dementia; Alzheimer's disease. Alternatively, in place of or in addition to methoxyflavones of formula (I), 3′,4′-dimethoxyflavone may be employed.

Yet another aspect of the present invention relates to an antioxidant (more specifically an in vivo oxidation preventing, suppressing or reducing agent) or a composition for preventing, suppressing or reducing in vivo oxidation that contains at least one methoxyflavone of formula (I) (in the subject specification, the terms “antioxidant”, “in vivo oxidation preventing, suppressing or reducing agent” and “composition for preventing, suppressing or reducing in vivo oxidation” are used interchangeably and unless otherwise specified, when one of these three terms is used, the remaining two terms shall also be intended.) In another mode, this invention relates to a method for preventing, suppressing or reducing in vivo oxidation which comprises administering at least one methoxyflavone of formula (I) to a subject. In the subject specification, the term “in vivo oxidation” means various oxidation reactions that are caused in a living body by active oxygen. Alternatively, in place of or in addition to methoxyflavones of formula (I), 3′,4′-dimethoxyflavone may be employed.

The total content of methoxyflavones of formula (I) in the NOX inhibitors, agents for preventing or treating NOX-associated diseases, and antioxidants according to the present invention are not particularly limited as long as the desired effects are obtained and it is preferably 0.01 to 50 w/w %, more preferably 0.1 to 40 w/w %, and even more preferably 0.5 to 30 w/w %.

The total intake by adult per day of the methoxyflavones of formula (I) for exhibiting the foregoing desired effects such as NOX inhibiting action, prevention or treatment of NOX-associated diseases, and antioxidation is preferably 1 to 500 mg, more preferably 3 to 200 mg, and even more preferably 5 to 100 mg.

Those quantitative values are also applicable to the case of using 3′,4′-dimethoxyflavone.

(NFκB Inhibitors)

The present inventor has found that methoxyflavones of formula (I) are effective as NFκB inhibitors. Therefore, another aspect of the present invention relates to a NFκB inhibitor or a composition for inhibiting NFκB that contains at least one methoxyflavone of formula (I) (in the subject specification, the terms “NFκB inhibitor” and “composition for inhibiting NFκB” are used interchangeably and unless otherwise specified, when one of these terms is used, the other term shall also be intended.) In another mode, this invention relates to a method for inhibiting NFκB that comprises administering at least one methoxyflavone of formula (I) to a subject. Alternatively, in place of or in addition to methoxyflavones of formula (I), 3′,4′-dimethoxyflavone may be employed.

Inhibiting NFκB leads to preventing or treating NFκB-associated diseases. Therefore, another aspect of the present invention relates to an agent for preventing or treating NFκB-associated diseases or a composition for preventing or treating NFκB-associated diseases that contains at least one methoxyflavone of formula (I) (in the subject specification, the terms “agent for preventing or treating NFκB-associated diseases” and “composition for preventing or treating NFκB-associated diseases” are used interchangeably and unless otherwise specified, when one of these terms is used, the other term shall also be intended.) In another mode, the present invention relates to a method for preventing or treating NFκB-associated diseases that comprises administering at least one methoxyflavone of formula (I) to a subject. Such diseases include: rheumatoid arthritis, inflammatory colitis, osteoarthritis, osteolysis, tendinitis, sciatica, herniated disc, stenosis, myelosis, low back pain, zygapophyseal joint pain, carpal tunnel syndrome, tarsal tunnel syndrome, failed back surgery syndrome, AIDS, arteriosclerosis, asthma, arthritis, diabetes, inflammatory colitis, hepatitis, stroke, dementia, muscle wasting, viral infection, skin aging including photoaging, cancer, and aging. Alternatively, in place of or in addition to methoxyflavones of formula (I), 3′,4′-dimethoxyflavone may be employed.

The total content of methoxyflavones of formula (I) in the NFκB inhibitors, and agents for preventing or treating NFκB-associated diseases according to the present invention are not particularly limited as long as the desired effects are obtained and it is preferably 0.01 to 50 w/w %, more preferably 0.1 to 40 w/w %, and even more preferably 0.5 to 30 w/w %.

The total intake by adult per day of methoxyflavones of formula (I) for exhibiting the foregoing desired effects such as NFκB inhibiting action and prevention or treatment of NFκB-associated diseases is preferably 1 to 500 mg, more preferably 3 to 200 mg, and even more preferably 5 to 100 mg.

Those quantitative values are also applicable to the case of using 3′,4′-dimethoxyflavone.

(Other Components)

The NOX or NFκB inhibitors, agents for preventing or treating NOX- or NFκB-associated diseases, and antioxidants according to the present invention may, in addition to methoxyflavones of formula (I), have any components incorporated as long as they will not compromise the intended effects. For example, not only physiologically active components including vitamins such as vitamin E and vitamin C, minerals, hormones, nutrients and fragrances but also emulsifiers, isotonization agents (tonicity agents), buffers, solubilizing agents, antiseptics, stabilizers and other additives that are incorporated in formulating procedures may also be incorporated.

(Applications)

The NOX or NFκB inhibitors, agents for preventing or treating NOX- or NFκB-associatd diseases, and antioxidants according to the present invention can be used as foods or beverages (e.g. functional foods, health supplements, foods with nutrient function claims, foods for special dietary uses, foods for specified health uses, nutritional supplements, foods for medical diet, health foods, dietary supplements, etc.), pharmaceuticals or cosmetics, or as starting materials therefor. The foods or beverages and pharmaceuticals may be pet foods, animal feeds, etc. that are processed as feeds for pets, as well as veterinary pharmaceuticals.

The form of said foods or beverages is not particularly limited and examples include soft drinks (e.g. sports drinks, carbonated drinks, fruit juice containing drinks), confectionery (e.g. cakes, biscuits, breads, candies), noodles (e.g. udon, soba, ramen, pasta), miso, soy sauce, vinegar, salad oil, sesame oil, soy milk, and cow milk. Other possible forms include tablets, granules, powders, capsules (including soft capsules), and so on.

The form of said pharmaceuticals is not particularly limited and examples include preparations for external application (e.g. lotions, emulsions, patches, ointments), oral preparations (tablets, granules, powders, capsules (including soft capsules), solutions, suspensions), injections, and infusions.

The form of said cosmetics is not particularly limited and examples include toilet waters, jells, lotions, creams, face masks, milk emulsions, foundations, lipsticks, powder rouges, facial washes, and hair tonics.

(Numerical Ranges)

For the purpose of clarity, it should be noted that whenever a numerical range is defined herein by its lower and upper limits and expressed as “lower limit to upper limit,” both the lower and upper limits are included. For example, the range expressed as “1 to 2” includes 1 and 2.

### EXAMPLES

### Example 1

### Isolation and Purification of Methoxyflavones

To 150 g of black ginger, 1500 ml of a 50% aqueous ethanol solution was added and the mixture was heated under reflux for 2 hours to perform extraction. After cooling, the resulting liquid extract was filtered, concentrated under reduced pressure and freeze-dried to yield 25.7 g of a black ginger extract. A 9 g portion of the resulting extract was subjected to column chromatography using Dia ion HP20 (product of Mitsubishi Chemical Corporation) and fractionated into four fractions (a portion eluted with 30% ethanol; a portion eluted with 50% ethanol; a portion eluted with 70% ethanol; and a portion eluted with 100% ethanol). The portion eluted with 50% ethanol was subjected to high-speed liquid chromatography to isolate 5,7,3′,4′-tetramethoxyflavone (64 mg), 3,5,7,3′,4′-pentamethoxyflavone (464 mg), 5,7-dimethoxyflavone (145 mg), 5,7,4′-trimethoxyflavone (188 mg), 3,5,7-trimethoxyflavone (35 mg), and 3,5,7,4′-tetramethoxyflavone (96 mg). Subsequently, the portion eluted with 100% ethanol was also subjected to the same procedures of separation and purification by liquid chromatography to isolate 5-hydroxy-3,7,3′,4′-tetramethoxyflavone (15 mg), 5-hydroxy-7-methoxyflavone (84 mg), 5-hydroxy-7,4′-dimethoxyflavone (56 mg), 5-hydroxy-3,7-dimethoxyflavone (100 mg), and 5-hydroxy-3,7,4′-trimethoxyflavone (110 mg). The isolated compounds were identified by comparing their spectrum data with the various spectrum data presented in a document (“The Structures of Components in A Plant of the Zingiberaceae Family, Kaempferia parviflora, as well as Their α-Glucosidase Inhibitory Activities and Antimutagenicities” which is a doctor's thesis of Mr. Toshiaki Azuma, Graduate School of Human Life Science, Osaka City University).

### Example 2

### Production of Oil or Fat Extracts

Two samples of black ginger (3 g and 15 g) were each mixed with 30 mL of olive oil and subjected to extraction at 120° C. for 30 minutes; thereafter, the mixtures were cooled and filtered to yield two pale yellow oil or fat extracts of black ginger. Under the analytical conditions set forth below, the two pale yellow oil or fat extracts were determined for their total content of the eleven methoxyflavones mentioned in Example 1; the respective values were 6.2 mg/mL (from the black ginger weighing 3 g) and 22.4 mg/mL (from the black ginger weighing 15 g). Each of the extracts contained all of the eleven methoxyflavones mentioned in Example 1.

(Analysis and Quantification of Methoxyflavones)

To 1.0 mL of a black ginger's oil or fat extract, 1.0 mL of n-hexane was added for dilution and, thereafter, three extractions of methoxyflavones were conducted with 2.0 mL of an 80% aqueous methanol solution. In each run, the resulting 80% liquid methanol extract was passed through Mega Bond Elute C18 (product of Agilent Technologies Japan, Ltd.), followed by passing 2.0 mL of 80% methanol with a view to washing out the methoxyflavones adsorbed on Mega Bond. Elute C18. The resulting liquids were combined and diluted to a final volume of 10 mL for use as a sample for analysis by HPLC.

(Conditions for HPLC Analysis)

Column: Develosil C30 UG5 (4.6×150 mm, 5 μm; product of Nomura Chemical Co., Ltd.)

Detection: 280 nm (200-600 nm for PDA detection)

Column temperature: 40° C.

Mobile phase A: 0.05% trifluoroacetic acid in aq. sol.

Mobile phase B: 0.05% trifluoroacetic acid solution in 90% acetonitrile in aq. sol.

Gradient: Mobile phase B 50%→50%→70%→70% (0 min→7.5 min→20 min→25 min)

Flow rate: 1.0 mL/min

Sample injection: 10 μL

### Example 3

### Method of Measuring NOX Inhibitory Activity

Preparing Differentiated HL-60 Cells:

Human myeloid leukemia cell HL-60 repeats proliferation in an undifferentiated state but upon addition of DMSO (dimethyl sulfoxide), retinoic acid or the like, it is known to differentiate into mature granulocytes and lose the ability to proliferate, as accompanied by intracellular expression of NOX (NADPH oxidase) which also serves as an index for differentiation; the expressed NOX can be utilized as an enzyme source for evaluating NOX inhibitory activity.

To induce differentiation into NOX expressing granulocytes, undifferentiated HL-60 cells cultured in a 10% FBS supplemented RPMI 1640 medium were suspended in a 1% DMSO containing, 10% FBS supplemented RPM 11640 medium to give a density of 5×10⁵ cells/ml, and the suspension was distributed among Petri dishes (i.d. 10 cm) in 15 ml portions and cultured in a CO₂ incubator (37° C.) for three days; thereafter, 10 ml of a 1% DMSO containing, 10% FBS supplemented RPM 11640 medium was added into each of the Petri dishes and culture was performed for an additional three days, thus yielding differentiated HL-60 cells in which NOX was expressed. As described below, the differentiated HL-60 cells, either as a homogenate or in a viable state, were subjected to NOX activity measurement.

NOX Activity Measurement in a Cell-Free System Using a Homogenate:

HL-60 cells differentiated by DMSO treatment were collected by centrifugation and, after being washed once with PBS (phosphate buffered physiological saline), suspended in a homogenizing buffer (8 mM phosphate buffered solution containing 131 mM NaCl and 340 mM sucrose; pH 7.0) to give a density of 1×10⁸ cells/ml. After being cooled with ice, the suspension was treated with a sonicator (Bioruptor UCD-250 HSA; product of Cosmo Bio Co., Ltd.) by repeating three cycles of a process under the condition of 4° C. or below that consisted of 20-sec disrupting at maximum power and 30-sec interval cooling, whereupon a cell homogenate was obtained. The homogenate was centrifuged at 1000 g for 4 minutes to remove debris; to the resulting supernatant, nine volumes of a reaction buffer (65 mM phosphate buffered solution containing 1 mM EGTA, 10 μM FAD and 170 mM sucrose; pH 7.0) were added to prepare a homogenate's supernatant for NOX activity measurement (equivalent to 1×10⁷ cells/ml).

For reaction with NOX, 50 μl of the above-described cell homogenate was poured into each well of a 96-well microplate and after adding 25 μl of a 0.5 mM SDS solution as a NOX activator and 25 μl of a 0.4 mM NADPH solution as a substrate, the reaction was carried out at 25° C. for 30-90 minutes. The NOX activity was determined by measuring the rate of NADPH consumption through fluorometry (Ex: 355 nm/Em: 460 nm).

To measure the NOX inhibitory activity of a test sample, a DMSO solution of the sample (usually 10 mM for a reagent) was prepared, from which 3-fold serial dilutions were prepared by adding DMSO; the dilutions were each added in a volume of 1 μl/well to the above-described reaction solution to thereby carry out an enzymatic reaction; the resulting inhibitory activity was indicated in terms of IC₅₀ value (in μM, or μg/ml for extract).

NOX Activity Measurement Using Viable, Differentiated HL-60 Cells:

HL-60 cells differentiated by DMSO treatment were collected by centrifugation and suspended in a FBS- and Phenol Red-free D-MEM medium to give a density of 5×10⁶ cells/ml. For reaction with NOX, 25 μl of the above-described cell suspension was poured into each well of a 96-well microplate and, furthermore, 0.8 mg/ml of a WST-1 solution as prepared using the above-described D-MEM and a test sample dissolving solution as prepared at a predetermined concentration (i.e., a DMSO solution of the sample (usually 10 mM for a reagent) was prepared, from which 3-fold serial dilutions were prepared using DMSO and added to the above-described D-MEM to give a concentration of 1 v/v % or below, thereby preparing the test sample dissolving solution of interest) were each added in a 25 μl portion, followed by stirring of the mixture; thereafter, 25 μl of 4 μM PMA (Phorbol 12-Myristate 13-acetate in a final concentration of 1 μM) as dissolved in D-MEM was added to activate NOX; reaction was carried out at 37° C. for 45 minutes, whereupon superoxide as the NOX enzymatic product reacted with WST-1 in the reaction solution to generate yellow formazan and its quantity was measured in terms of absorbance at 450 nm. It should be noted that in this system of NOX activity measurement, NOX would not be activated unless PMA was added.

The resulting inhibitory activity was indicated in terms of IC₅₀ value (in μM, or μg/ml for extract).

### Example 4

### Comparison of Methoxyflavonoids for NOX Inhibitory Activity

A number of methoxyflavonoids were measured for their NOX inhibitory activity by performing NOX activity measurements in a cell-free system using a homogenate in accordance with Example 3. The results are shown in Table 1.

As Table 1 shows, strong NOX inhibitory activity was observed in 5,7,3′,4′-tetramethoxyflavone which was a methoxy form of luteolin, and in 3,5,7,3′,4′-pentamethoxyflavone which was a methoxy form of quercetin. These 5,7-dimethoxyflavonoids are contained typically in black ginger. On the other hand, hexamethoxyflavones such as nobiletin that are known to occur in citrus fruits were not found to have any strong NOX inhibitory activity. It should be noted here that the NOX inhibitor VAS 2870 had an IC₅₀ of 3.3 μM in the cell-free system using a homogenate.

TABLE 1

| Chemicals | | IC50 (μM) |
|---|---|---|
| 5,6,7,8,3′,4′-Hexamethoxyflavone (Nobiletin) | Flavone | 80 |
| 5,6,7,3′,4′,5′-Hexamethoxyflavone | Flavone | ~100 |
| 5,7,3′,4′,5′-Pentamethoxyflavone | Flavone | >100 |
| 7,8,3′,4′-Tetrahydroxyflavone | Flavone | ~100 |
| 7,8,3′,4′-Tetramethoxyflavone | Flavone | ~100 |
| 5,7,3′-Trihydroxy-3,4′-dimethoxyflavone | Flavone | ~100 |
| 5,7-Dihydroxy-3′,4′,5′-trimethoxyflavanone | Flavanone | >>100 |
| 2′,6′-Dihydroxy-4,4′-dimethoxydihydrochalcone | Chalcone | >>100 |
| 2′,6′-Dihydroxy-4′-methoxychalcone | Chalcone | >>100 |
| 2,3-Dimethoxy-2′-hydroxychalcone | Chalcone | >>100 |
| 4-Methoxychalcone | Chalcone | >>100 |
| 4′-Methoxychalcone | Chalcone | >>100 |
| 3,5,7,3′,4′-Pentamethoxyflavone (Quercetin- | Flavonol | 23 |
| 3,5,7,3′,4′-pentamethylether) | | |
| 3,7,3′,4′-tetramethoxyflavone (Quercetin-3,7,3′,4′- | Flavonol | >100 |
| tetramethylether) | | |
| 3′,4′-Dihydroxyflavone | Flavone | 75 |
| 3′,4′-Dimethoxyflavone | Flavone | 15 |
| 5-Methyl-7-methoxy-isoflavone | Isolavone | >100 |
| 5,7,3′,4′-Tetramethoxyflavone (Luteolin | Flavone | 8 |
| tetramethylether) | | |
| o-Acetylcolumblanetin | Coumarin | 19 |
| 3-Acetylcoumarin | Coumarin | >>100 |
| 8-Acetyl-6,7-dimethoxycoumarin | Coumarin | >100 |
| 8-Acetyl-7-hydroxycoumarin | Coumarin | >100 |
| 8-Acetyl-6-hydroxy-7-methoxycoumarin | Coumarin | >>100 |
| 8-Acetyl-7-methoxycoumarin | Coumarin | >>100 |
| VAS2870 (NOX inhibitor) | | 3.3 |

### Example 5

### NOX Inhibitory Activity of Compounds Derived from Black Ginger

Methoxyflavonoids extracted and fractionated from black ginger were measured for their NOX inhibitory activity by performing NOX activity measurements in a cell-free system using a homogenate in accordance with Example 3. Each of the flavonoids obtained from black ginger is characterized by having methoxy groups. Among these floavonoids, flavones having a methoxy group at positions 5 and 7 of the A ring were found to have strong NOX inhibitory activity but flavonoids having a hydroxyl group at position 5 was not found to have any NOX inhibitory activity. The methoxy group at position 3 was found to have an activity attenuating, though not fatal, tendency. It is therefore assumed that methoxyflavones represented by formula (I) have superior NOX inhibitory activity. VAS 2870 (NOX inhibitor) had an IC₅₀ of 6.3 μM in the system under consideration.

TABLE 2

| | | NOX | Extract's | NOX inhibitory |
|---|---|---|---|---|
| | | inhibition | yield | activity |
| Fractions | Identified compounds | IC50 (μM) | (wt %) | (distribution %) |
| KPEt-21 | 5,7,3′,4′-Tetramethoxyflavone | 38 | 0.04 | 12 |
| | (Luteolin tetramethylether) | | | |
| KPEt-22 | 3,5,7,3′,4′-Pentamethoxyflavone | 70 | 0.31 | 45 |
| | (Quercetin-3,5,7,3′,4′- | | | |
| | pentamethylether) | | | |
| KPEt-23 | 5,7-Dimethoxyflavone | 53 | 0.10 | 19 |
| KPEt-25 | 5,7,4′-Trimethoxyflavone | 53 | 0.13 | 21 |
| KPEt-261 | 3,5,7-Trimethoxyflavone | >100 | 0.02 | — |
| KPEt-262 | 3,5,7,4′-Tetramethoxyflavone | >>100 | 0.06 | — |
| KPEt-41 | 5-hydroxy-3,7,3′,4′- | >100 | 0.01 | — |
| | Tetramethoxyflavone | | | |
| KPEt-421 | 5-hydroxy-7-Methoxyflavone | >>100 | 0.06 | — |
| KPEt-422 | 5-hydroxy-7,4′-Dimethoxyflavone | >>100 | 0.04 | — |
| KPEt-431 | 5-hydroxy-3,7-Dimethoxyflavone | >>100 | 0.07 | — |
| KPEt-432 | 5-hydroxy-3,7,4′-Trimethoxyflavone | >>100 | 0.07 | — |
| VAS 2870 | | 6.3 | | |
| (NOX | | | | |
| inhibitor) | | | | |

### Example 6

To check for the difference that might be caused to the composition of fat or oil extracts from different lots of black ginger, two black ginger samples each weighing 200 g were provided and to each sample, 1000 mL of ethanol was added and the mixture was heated under reflux for an hour to perform extraction. The resulting liquid was cooled and thereafter filtered by suction to be separated into the residue and the liquid extract. To the residue, 1000 mL of ethanol was added again and the mixture was heated under reflux for an hour to perform extraction; after filtration, the resulting liquid extract was combined with the previously obtained liquid extract. Subsequently, 100 mL of middle-chain fatty acid triglyceride was added to the combined liquid extracts and after distilling off ethanol by concentrating under reduced pressure, the precipitating insoluble matter was removed through filtration by suction to yield two oil or fat extracts of black ginger. When the contents of methoxyflavones in these extracts were analyzed as in Example 2, the total amount of methoxyflavones was 90.4 mg/mL (in the extract which is hereinafter referred to as Extract A) and 54.9 mg/mL (in the extract which is hereinafter referred to as Extract B). Using olive oil, the total amount of methoxyflavones in each of these two extracts was adjusted to 5 mg/ml; the thus obtained two solutions were measured for absorbance at 660 nm, which was 0.036 (in Extract A) and 0.030 (in Extract B), with methanol being used as a blank. It should be noted that each of these extracts contained all of the eleven methoxyflavones mentioned in Example 1.

### Example 7

### Preparing Samples for NOX Inhibitory Activity Measurement

### Production of Black Ginger's Ethanol Extracts

To 200 g of dried black ginger, 1000 mL of a 50% aqueous ethanol solution was added and the mixture was heated under reflux for an hour to perform extraction. The resulting liquid was cooled and thereafter filtered by suction to be separated into the residue and the liquid extract. To the residue, 1000 mL of a 50% aqueous ethanol solution was added again and the mixture was heated under reflux for an hour to perform extraction; after filtration, the resulting liquid extract was combined with the previously obtained liquid extract. After cooling to room temperature, the combined liquid extracts were concentrated under reduced pressure and then freeze-dried to yield black ginger's ethanol extract No. 1 in an amount of 49 g (yield: 24.5%). To check for the difference due to different lots of black ginger, the same procedure as described above was taken to yield black ginger's ethanol extract No. 2 in an amount of 23 g (yield: 15.2%). Subsequently, the total content of eleven methoxyflavones in these extracts was analyzed substantially in accordance with the method of Example 2; the respective values were 264 mg/g and 267 mg/g. Using methanol, the total amount of methoxyflavones in black ginger's ethanol extract No. 1 was adjusted to 5 mg/ml; the thus obtained solution was measured for absorbance at 660 nm, which was 0.95 (the blank was methanol). It should be noted that each of these extracts contained all of the eleven methoxyflavones mentioned in Example 1.

### Example 8

### Preparing Samples for NOX Inhibitory Activity Measurement

### Production of Black Ginger's Oil or Fat Extracts

To each of black ginger samples weighing 10 g, 20 g, 30 g and 40 g, 10 volumes of ethanol were added and the mixture was heated under reflux for an hour to perform extraction. The resulting liquid was cooled and then filtered by suction; to the resulting liquid extract, 15 mL of middle-chain fatty acid triglyceride was added and ethanol was distilled off by concentrating under reduced pressure; thereafter, with a view to removing the insoluble matter, filtration by suction was performed again, yielding four black ginger's oil or fat extracts. The thus obtained four black ginger's oil or fat extracts were analyzed for the total content of eleven methoxyflavones substantially in accordance with Example 2; the respective values were 23.9 mg/mL, 46.3 mg/mL, 69.4 mg/mL, and 78.1 mg/mL. When the oil or fat extracts containing the methoxyflavones at a concentration of 46.3 mg/mL or more were left to stand at room temperature, methoxyflavones were found to precipitate. It should be noted that each of these extracts contained all of the eleven methoxyflavones mentioned in Example 1.

### Example 9

### Comparison of Black Ginger Extraction Methods Using NOX Inhibitory Activity as an Index

In accordance with Example 3, black ginger's oil or fat extract No. 1 with a total methoxyflavone content of 22.4 mg/ml (as obtained in Example 2), black ginger's oil or fat extract No. 2 with a total methoxyflavone content of 69.4 mg/ml (as obtained in Example 8) and an ethanol extract (black ginger's ethanol extract Nos. 1 and 2 as obtained in Example 7) were measured for NOX inhibitory activity (NOX inhibitory activity measurement using viable, differentiated HL-60 cells). To check for the difference in activity between lots of black ginger, investigation was made using two types of black ginger. The oil or fat extracts as just prepared would defy activity measurement, so they were degreased; specifically, 0.5 mL of an oil or fat extract was diluted with the same quantity (0.5 mL) of added n-hexane and, thereafter, methoxyflavones were extracted three times with 0.5 mL of an 80% aqueous methanol solution; the resulting liquid extract was adsorbed on Sep-Pak PLUS C8 125 Å Cartridges (product of Waters), through which 3.0 mL of 80% methanol was passed to remove the oil content. Thereafter, Sep-Pak PLUS C8 125 Å Cartridges was washed with a solvent and the resulting liquid was concentrated under reduced pressure and freeze-dried to prepare samples for evaluation. The measured IC₅₀ values are shown in Table 3 below.

The IC₅₀ values shown below represent the NOX inhibitory activity based on the total methoxyflavone content. Upon comparison of those values, the oil or fat extracts were both shown to have higher action (lower IC₅₀) than the ethanol extracts. This suggested that methoxyflavones having higher inhibitory action on NOX were extracted efficiently in the present invention by extraction with oils or fats.

TABLE 3

| | NOX IC₅₀ (μg/ml) |
|---|---|
| | Calculated for the total |
| Extract | methoxyflavone content |
| black ginger's ethanol extract No. 1 | 11.7 |
| black ginger's oil or fat extract No. 1 | 9.2 |
| black ginger's ethanol extract No. 2 | 7.7 |
| black ginger's oil or fat extract No. 2 | 3.1 |

### Example 10

### NFκB Inhibiting Action

It is known that when macrophage-like RAW 264.7 cells are stimulated with LPS (lipopolysaccharide), NFκB is activated, whereby the expression of iNOS (inducible NO synthetase) is enhanced, causing nitrous acid to accumulate in the culture broth due to the enzymatic activity of iNOS; the activation of NFκB can therefore be evaluated by measuring the buildup of nitrous acid in the culture broth.

RAW 264.7 cells were cultured in a 10% FBS supplemented RPMI 1640 culture broth. The cells were then suspended in the same culture broth to give a density of 4×10⁵ cells/ml; the suspension was distributed on a 96-well microplate in 100 μl portions per well and subjected to pre-culture (in a CO₂ incubator) for 24 hr. To the incubated suspension, a 6 ng/ml LPS containing culture broth (LPS was derived from E. coli and had a final concentration of 1 μg/ml) was added in a volume of 25 μl per well, and a solution of a test sample at a predetermined concentration was added in a volume of 25 μl per well; after culture for another 24 hr, the cell culture broth was recovered in divided 75 μl portions, to which an equal volume of Griess reagent (product of Fluka) was added to initiate a color reaction and the production of nitrous acid was measured on the basis of absorbance at 540 nm. To measure NFκB inhibitory activity, a DMSO solution of each test sample was dissolved in the foregoing culture broth to make 3-fold serial dilutions having DMSO concentrations of 1% and less and absorbance was measured with and without adding the stimulant LPS. The values of IC₅₀ (μM), or the concentration at which the production of nitrous acid due to LPS stimulation was inhibited by 50%, are indicated in Table 4. As is clear from Table 4, methoxyflavones of formula (I) showed superior NFκB inhibiting action.

TABLE 4

| | | NFκB inhibition |
|---|---|---|
| Fractions | Identified compounds | IC₅₀ (μM) |
| KPEt-21 | 5,7,3′,4′-Tetramethoxyflavone | 6 |
| KPEt-22 | 3,5,7,3′,4′-Pentamethoxyflavone | 32 |
| KPEt-23 | 5,7-Dimethoxyflavone | 7 |
| KPEt-25 | 5,7,4′-Trimethoxyflavone | 7 |
| KPEt-261 | 3,5,7-Trimethoxyflavone | 27 |
| KPEt-262 | 3,5,7,4′-Tetramethoxyflavone | 32 |
| KPEt-41 | 5-hydroxy-3,7,3′,4′-Tetramethoxyflavone | 55 |
| KPEt-421 | 5-hydroxy-7-Methoxyflavone | 42 |
| KPEt-422 | 5-hydroxy-7,4′-Dimethoxyflavone | 56 |
| KPEt-431 | 5-hydroxy-3,7-Dimethoxyflavone | 29 |
| KPEt-432 | 5-hydroxy-3,7,4′-Trimethoxyflavone | 30 |

### Example 11

### Relation Between Extraction Method and Composition

In view of the results of Example 9, compositional comparison was made between two types of extracts from black ginger, one obtained by extraction with oil or fat and another obtained by extraction with a hydrophilic solvent. Specifically, extraction with oil or fat was conducted substantially in accordance with Examples 2, 6 and 8 (extraction with oil or fat only, or extraction with ethanol followed by extraction with oil or fat) and extraction with ethanol was conducted substantially in accordance with Example 7. In the oil or fat extraction, olive oil or a mixture of olive oil with middle-chain fatty acid glyceride was used (in Example 11, a middle-chain fatty acid triglyceride was used, as sometimes denoted by MCT). The resulting extracts were analyzed by HPLC based on the method described in Example 2 and the obtained peak area data are shown below. In the following Tables, oil or fat is sometimes simply referred to as Oil for the sake of convenience.

TABLE 5A

| | Extraction method | | | | | | | | | |
|---|---|---|---|---|---|---|---|---|---|---|
| Methoxyflavones | EtOH [symbol] Oil | | EtOH | | Oil | | | | | |
| KPEt-21 | 38.2 | 6.5 | 11.2 | 28.9 | 19.1 | 23.7 | 11.8 | 10.2 | 7.4 | 7.2 |
| KPEt-22 | 207.2 | 36.2 | 57.8 | 134.2 | 95.4 | 119.5 | 72.3 | 62.9 | 48.1 | 64.7 |
| KPEt-23 | 633.8 | 129.1 | 69.2 | 145.0 | 103.1 | 127.5 | 137.1 | 119.7 | 67.7 | 65.7 |
| KPEt-25 | 391.8 | 71.9 | 82.5 | 187.6 | 132.0 | 164.0 | 108.6 | 94.4 | 68.4 | 66.3 |
| KPEt-261, 2 | 289.1 | 56.7 | 49.5 | 109.7 | 81.3 | 106.2 | 82.0 | 71.6 | 46.6 | 45.1 |
| KPEt-41 | 16.9 | 3.4 | 6.6 | 13.9 | 9.0 | 10.4 | 7.3 | 6.4 | 4.6 | 4.3 |
| KPEt-421 | 42.9 | 8.5 | 14.3 | 35.0 | 15.9 | 17.2 | 14.8 | 13.1 | 8.4 | 7.9 |
| KPEt-422 | 22.9 | 4.2 | 8.4 | 20.8 | 10.6 | 12.1 | 9.9 | 8.7 | 6.1 | 5.8 |
| KPEt-431 | 48.3 | 9.9 | 19.1 | 47.0 | 17.3 | 19.7 | 18.7 | 16.0 | 10.0 | 9.4 |
| KPEt-432 | 41.9 | 8.2 | 19.7 | 45.6 | 19.3 | 22.1 | 21.2 | 17.6 | 12.2 | 11.5 |
| A | 1271.0 | 243.7 | 220.8 | 495.7 | 349.6 | 434.6 | 329.8 | 287.2 | 191.6 | 185.9 |
| A + B | 1732.9 | 334.5 | 338.5 | 767.6 | 503.0 | 622.3 | 483.7 | 420.5 | 279.4 | 269.8 |
| A/(A + B) | 0.73 | 0.73 | 0.65 | 0.65 | 0.69 | 0.70 | 0.68 | 0.68 | 0.69 | 0.69 |
| A′ | 1063.75 | 207.47 | 162.98 | 361.48 | 254.20 | 315.14 | 257.48 | 224.29 | 143.37 | 139.19 |
| A′ + B′ | 1732.90 | 334.51 | 338.43 | 767.57 | 503.03 | 622.32 | 483.67 | 420.51 | 279.39 | 269.77 |
| A′/(A′ + B′) | 0.61 | 0.62 | 0.48 | 0.47 | 0.51 | 0.51 | 0.53 | 0.53 | 0.51 | 0.52 |

TABLE 5B

| | Extraction method | | | | | | | | |
|---|---|---|---|---|---|---|---|---|---|
| | Oil (containing MCT) | | | | | | | | |
| Methoxyflavones | 33% | 50% | 67% | 33% | 50% | 67% | 33% | 50% | 67% |
| KPEt-21 | 6.8 | 4.2 | 6.6 | 17.7 | 23.6 | 22.4 | 25.6 | 27.1 | 25.3 |
| KPEt-22 | 39.6 | 24.0 | 36.6 | 105.0 | 137.6 | 128.6 | 150.7 | 156.2 | 145.1 |
| KPEt-23 | 137.4 | 87.8 | 131.7 | 354.2 | 453.4 | 421.1 | 474.5 | 470.9 | 443.0 |
| KPEt-25 | 75.8 | 48.1 | 72.4 | 198.1 | 256.9 | 242.6 | 277.6 | 285.1 | 266.8 |
| KPEt-261, 2 | 62.2 | 37.9 | 57.2 | 162.0 | 207.2 | 190.5 | 220.9 | 220.4 | 205.6 |
| KPEt-41 | 4.1 | 2.3 | 3.4 | 10.1 | 13.1 | 11.5 | 14.7 | 15.3 | 13.1 |
| KPEt-421 | 10.3 | 5.8 | 8.6 | 27.6 | 35.1 | 30.2 | 38.6 | 42.1 | 32.7 |
| KPEt-422 | 5.2 | 2.8 | 4.3 | 13.9 | 17.8 | 16.2 | 20.7 | 22.6 | 17.7 |
| KPEt-431 | 12.2 | 6.8 | 10.0 | 32.1 | 40.4 | 35.7 | 44.5 | 48.7 | 37.1 |
| KPEt-432 | 10.0 | 5.5 | 8.0 | 26.5 | 33.6 | 30.3 | 38.7 | 42.1 | 32.7 |
| A | 259.6 | 164.1 | 247.3 | 675.0 | 871.4 | 814.7 | 928.5 | 939.3 | 880.2 |
| A + B | 363.6 | 225.1 | 338.8 | 947.2 | 1218.7 | 1129.1 | 1306.5 | 1330.4 | 1219.1 |
| A/(A + B) | 0.71 | 0.73 | 0.73 | 0.71 | 0.72 | 0.72 | 0.71 | 0.71 | 0.72 |
| A′ | 220.00 | 140.09 | 210.63 | 569.94 | 733.84 | 686.17 | 777.74 | 783.18 | 735.07 |
| A′ + B′ | 363.58 | 225.11 | 338.75 | 947.21 | 1218.71 | 1129.12 | 1306.50 | 1330.40 | 1219.11 |
| A′/(A′ + B′) | 0.61 | 0.62 | 0.62 | 0.60 | 0.60 | 0.61 | 0.60 | 0.59 | 0.60 |

*Percentages in the table represent the relative contents of MCT in the oil or fat used in extraction.

As is clear from Tables 5A and 5B, the oil or fat extracts had higher values of A/(A+B) and A′/(A′+B′), or higher proportions of methoxyflavones with high NOX or NFκB inhibitory activity, than the ethanol extracts. Such differences in composition can affect the NOX or NFκB inhibitory activity.

1. NOX inhibitor containing at least one methoxyflavone having a structure represented by the following formula (I): 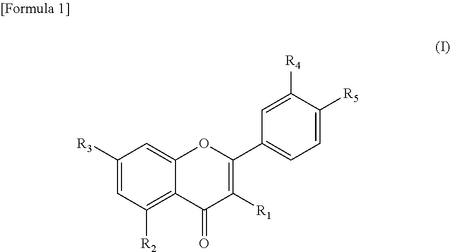
(where R₁, R₄ and R₅ are each independently hydrogen or a methoxy group, and R₂ and R₃ are each a methoxy group).

2. The NOX inhibitor according to claim 1, wherein the at least one methoxyflavone is selected from group A consisting of 5,7,3′,4′-tetramethoxyflavone, 3,5,7,3′,4′-pentamethoxyflavone, 5,7-dimethoxyflavone, and 5,7,4′-trimethoxyflavone.

3. The NOX inhibitor according to claim 2, wherein a ratio of the total content of said methoxyflavones of group A to the total contents of said methoxyflavones of group A and methoxyflavones of group B consisting of 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which is expressed as A/(A+B), exceeds 0.65 on a molar basis.

4. An agent for preventing or treating an NOX-associated disease that contains said at least one methoxyflavone as defined in claim 1.

5. The agent according to claim 4, wherein the NOX-associated disease is selected from the group consisting of allergic diseases, Parkinson's disease, cerebral infarction, cataract, epilepsy, spinal cord injury, arteriosclerosis, retinopathy of prematurity, renal disorder, peptic ulcer, pancreatitis, ulcerative colitis, myocardial infarction, adult respiratory distress syndrome, pulmonary emphysema, collagen diseases such as chronic rheumatoid arthritis, angiitis, edema, complications of diabetes, ultraviolet disorders, altitude sickness, porphyria, burns, frostbite, contact dermatitis, shock, failure of multiple organs, DIC, cancer, aging, fatigue, sarcopenia (progressive decline in skeletal muscle mass), mitochondrial dysfunction, dementia, and Alzheimer's disease.

6. The agent according to claim 4, wherein a ratio of the total content of said methoxyflavones of group A to the total contents of said methoxyflavones of group A and said methoxyflavones of group B, which is expressed as A/(A+B), exceeds 0.65 on a molar basis, and wherein group A methoxyflavones is selected from the group consisted of: 5,7,3′,4′-tetramethoxyflavone, 3,5,7,3′,4′-pentamethoxyflavone, 5,7-dimethoxyflavone, and 5,7,4′-trimethoxyflavone, and group B methoxyflavones is selected from the group consisting of: 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone.

7. NFκB inhibitor containing at least one methoxyflavone having a structure represented by the following formula (I): 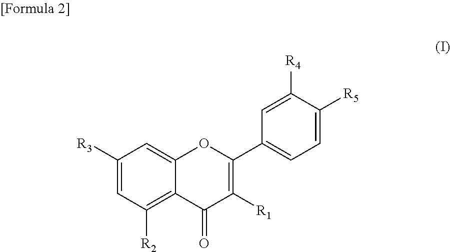
(where R₁, R₄ and R₅ are each independently hydrogen or a methoxy group, and R₂ and R₃ are each a methoxy group).

8. The NFκB inhibitor according to claim 7, wherein the at least one methoxyflavone is selected from group A′ consisting of 5,7,3′,4′-tetramethoxyflavone, 5,7-dimethoxyflavone, and 5,7,4′-trimethoxyflavone.

9. The NFκB inhibitor according to claim 8, wherein a ratio of the total content of said methoxyflavones of group A′ to the total contents of said methoxyflavones of group A′ and methoxyflavones of group B′ consisting of 3,5,7,3′,4′-pentamethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which is expressed as A′/(A′+B′), exceeds 0.48 on a molar basis.

10. An agent for preventing or treating an NFκB-associated disease that contains said at least one methoxyflavone as defined in claim 7.

11. The agent according to claim 10, wherein the NFκB-associated disease is selected from the group consisting of rheumatoid arthritis, inflammatory colitis, osteoarthritis, osteolysis, tendinitis, sciatica, herniated disc, stenosis, myelosis, low back pain, zygapophyseal joint pain, carpal tunnel syndrome, tarsal tunnel syndrome, failed back surgery syndrome, AIDS, arteriosclerosis, asthma, arthritis, diabetes, inflammatory colitis, hepatitis, stroke, dementia, muscle wasting, viral infection, skin aging including photoaging, cancer, and aging.

12. The agent according to claim 10, wherein a ratio of the total content of said methoxyflavones of group A′, wherein group A′ methoxyflavones are selected from the group consisting of: 5,7,3′,4′-tetramethoxyflavone, 5,7-dimethoxyflavone, and 5,7,4′-trimethoxyflavone, to the total contents of said methoxyflavones of group A′ and said methoxyflavones of group B′, wherein Group B′ methoxyflavones are selected from the group consisting of 3,5,7,3′,4′-pentamethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which is expressed as A′/(A′+B′), exceeds 0.48 on a molar basis.

13. NOX inhibitor containing 3′,4′-dimethoxyflavone.

14. An agent for preventing or treating an NOX-associated disease that contains 3′,4′-dimethoxyflavone.

15. The agent according to claim 14, wherein the NOX-associated disease is selected from the group consisting of allergic diseases, Parkinson's disease, cerebral infarction, cataract, epilepsy, spinal cord injury, arteriosclerosis, retinopathy of prematurity, renal disorder, peptic ulcer, pancreatitis, ulcerative colitis, myocardial infarction, adult respiratory distress syndrome, pulmonary emphysema, collagen diseases such as chronic rheumatoid arthritis, angiitis, edema, complications of diabetes, ultraviolet disorders, altitude sickness, porphyria, burns, frostbite, contact dermatitis, shock, failure of multiple organs, DIC, cancer, aging, fatigue, sarcopenia (progressive decline in skeletal muscle mass), mitochondrial dysfunction, dementia, and Alzheimer's disease.
